(19) United States
(12) Patent Application Publication
(10) Pub. No.: US 2004/0166504 A1
(43) Pub. Date: Aug. 26, 2004

(54) Microfluidic chemical assay apparatus and method

(76) Inventors: Joel Stephane Rossier, Saillon (CH); Frederic Reymond, La Conversion (CH); Philippe Michel, Collombey (CH)

Correspondence Address: HOWSON AND HOWSON, ONE SPRING HOUSE CORPORATION CENTER, BOX 457, 321 NORRISTOWN ROAD, SPRING HOUSE, PA, 19477, US

(21) Appl. No.: 10/481,152

(22) PCT Filed: Jul. 4, 2002
(86) PCT No.: PCT/IB02/03220

(30) Foreign Application Priority Data
Jul. 4, 2001 (GB) 0116384.9

(51) Int. Cl. C12Q 1/68

(52) U.S. Cl. 435/6

(57) ABSTRACT

Apparatus method for performing an electrochemical assay or a reaction, using electrical conductivity and/or power in order either to perform a reduction or an oxidation or an ion transfer reaction, or to perform conductimetry and/or impedance measurements, or to generate an electric field in a solution, or to perform any combination of the aforesaid. The apparatus comprises at least one micro-chip (1) possessing a microstructure (2) (for example a microchannel or array or network of microchannels) having a tip end (3) adapted for uptake of a fluid sample into and/or discharge of a fluid sample from said microstructure, a microfluidic connection end (4) and an integral electrode. It also comprises a microfluidic control unit (11) communicating with the microfluidic connection end of the microstructure and adapted to push, pull or block fluids in the microstructure, and an electrochemical unit adapted to apply an electric field or a current to fluid in the microstructure and/or to measure an electrochemical event therein. Optionally, there is support means adapted to support the micro-chip(s) in relation to the microfluidic control unit in such a manner as to ensure fluid-tight connection therebetween.

### FIELD OF THE INVENTION

This invention relates to apparatus and methods for performing fully or semi-automated electrochemical assays or reactions in micro fluidic chips.

### BACKGROUND OF THE INVENTION

In recent years, the miniaturisation of analytical chemical and biochemical tools has become an expanding field. The main factors encouraging the development of miniaturised chemical apparatus are the desire for decreased analyte consumption, rapid analysis and improved automation capacity. These needs are particularly evident in the field of life sciences, where biomedical diagnostics, genetic analysis, proteomics and high throughput screening in drug discovery are becoming increasingly important. The need to limit analyte consumption is highlighted by the increasing number of assays that are performed, the use of reactants for analysis requiring to be kept as small as possible in order not only to reduce costs but also to limit waste production. In the case of biomedical diagnostics, the analysis of extremely small volumes is often required and the minimisation of analysis time is desirable, as are simplified handling procedures that decrease manipulations and minimise cross-contamination from sample to sample. Previously, two different but complementary strategies have been investigated for achieving these goals: microfluidic devices and high density 2-D arrays with immobilized affinity reagents.

In the field of micro-analytical systems, a very important issue for the development of true operational devices is the automation of the assays, since the reproducibility of the measurements as well as the number of analyses that can be performed can thus be significantly improved. For the automation of measurements using Microsystems, the most critical point is probably the reagent dispensing system. Until now, some automated devices have been developed for micromethods based on highly dense parallel networks, such as arrays of microspots or microwells. In these cases, the delivery systems are generally composed of one or several needles allowing the aspiration and the dispensing of the required volumes of reagents at very precise points. In the case of microfluidic systems, an additional key problem for the automation of measurements is the filling of microchannels and controlling the movement of reagents within them. Microfluidic automated devices based on capillary electrophoresis have been developed in the past, for example a full DNA analyser was implemented in a single device with a polymerase chain reaction chamber followed by electrophoretic separation.

Some automated analytical methods in which micropipette tips are used both as the reaction solid phases and for reagent handling have been previously developed. This was done by immobilising biomolecules, such as antibodies, on the walls of the tips and by using these tips to pipette the reagents. Using this kind of approach, the contamination risks from sample to sample can be limited. The connection of the microfluidic devices with external sample solution has been addressed by different means, such as connecting the microfluidic chip to a capillary and then dipping the capillary in the sample solution and pumping the solution inside the microchip by electroosmotic flow (WO 00/21666). In other cases, the chip is connected to a number of microsyringe pumps so as to deliver the sample inside the microchip (WO 01/63270). Some devices have used pulses to let the sample enter the chip with gas or high voltage (U.S. Pat. No. 6,395,232). Others have used capillary fill from a needle etched channel tip to have their channel sampled by capillary action and to perform electrochemical assays such as glucose detection (Sensor Actuator A Vol 95, 2002, 108-113). Such method does not enable any control of the fluidics within the channel.

When performing analytical assays it is of prime importance to control the flow rate during sample delivery. Indeed, due to the very small volume of the channel (in the order of picolitres to microlitres) small variations in the sample flow rate induce dramatic variation in the volume that is transferred through the channel. If a reaction involves immunosorption or physisorption for instance, sever deviation of the detection can occur for the same sample concentration. For this reason, the present invention aims to control and monitor the flow of the sample solution by electrochemical means.

### SUMMARY OF THE INVENTION

The present invention provides an apparatus and related methods for performing fully automated or semi-automated assays or reactions in microchips. The microchips include microchannels or microchannel arrays or networks, enabling handling of sample and reagents as well as achievement of reactions followed by electrochemical events. They can also be used for reagent handling only, for instance in the case where the present apparatus is used to uptake or dispense fluids from a micro-chip.

More specifically, the invention provides, in one aspect, apparatus for performing an electrochemical assay or a reaction, using electrical conductivity and/or power in order either to perform a reduction or an oxidation or an ion transfer reaction, or to perform conductimetry and/or impedance measurements, or to generate an electric field in a solution, or to perform any combination of the aforesaid, the apparatus comprising: at least one micro-chip, the or each said micro-chip possessing at least one microstructure having: a tip end adapted for uptake of a fluid sample into and/or discharge of a fluid sample from said microstructure; a microfluidic connection end; and an integral electrode; a microfluidic control unit communicating with said microfluidic connection end of said microstructure and adapted to push, pull or block fluids in said microstructure; an electrochemical unit adapted to apply an electric field or a current to fluid in said microstructure and/or to measure an electrochemical event therein; and, optionally, support means adapted to support said micro-chip(s) in relation to said microfluidic control unit in such a manner as to ensure fluid-tight connection therebetween.

The invention provides, in another aspect a method of performing an electrochemical assay or a reaction, using the apparatus of any preceding claim, the method comprising the steps of: (a) placing said microchip in said support means; (b) placing a sample in contact with said microstructure tip; (c) filling said microstructure with said sample, either by capillary action or by pumping or aspirating said sample by means of said microfluidic control unit; (d) using said microfluidic control unit either to pull, push or block said sample in said microstructure; (e) actuating said electrochemical unit to perform an electrochemical assay using electrical conductivity and/or power to perform a reduction or an oxidation or an ion transfer reaction, or to perform conductimetry and/or impedance measurements, or to generate an electric field in a solution, or to perform any combination of the aforesaid; and (f) optionally, repeating steps (b) to (e).

Generally, the microchips incorporate sealed microchannels with two apertures (one at each extremity) and they can be fabricated using different materials including conductive ones for their use in electrochemical assays.

One or several individual or interconnected microchips can be fabricated individually and/or on the same support. They can be used individually or as an array of independent or interconnected microstructures.

Preferably, the lower extremity of the microchip incorporates at least one tip connected to the microchannel(s) that will be placed in contact with the sample solution to be analysed or to react. The upper part of the microchip preferably contains an outlet for the microchannel(s) that can be connected with an automated microfluidic control device allowing filling and/or emptying of the microchannels. In some embodiments, the fluidic control device may be a simple micropipette for mechanical pumping. Preferably, the microchips are capable of displacement (e.g. sequential displacement) in x, y and/or z directions, either by automated means or manually.

The control of the flow in the microstructure during the sampling is important to enable reproducible results. For this reason, it is preferred that the apparatus incorporate an integral electrode for monitoring the fluid flow in the microstructure. It is well known to use an electrode not only for detecting if a channel is filled or empty but also for measuring the flow of solution by amperometry. Conductivity detection may be utilised to measure the time required for the solution to cross the microstructure. This can be done by having different electrode pairs at the entrance, at different places along the microstructure and at the inlet or outlet of the microstructure. Fluidic control can be performed by monitoring the flow rate by means of amperometrical detection, it having been demonstrated previously that the detected current depends upon the flow rate according to the Ilkowich equation:

I=0.925nFcL(ID)^{2/3}(Fv/h²d)^{1/3}

Where I is the current, n the number of electrons exchanged per oxidised molecule, L the width of the electrode, l the length of the electrode, D the diffusion coefficient of the oxidised molecule, Fv the flow rate, h the half-height of the channel, d the with of the channel.

It is notable that this kind of electrochemical measurement may be quantitative (i.e. when amperometry is used to monitor the concentration of an electroactive species). Therefore, the signal measured during the sample loading, during the various steps of an assay (incubation, washing, etc.) or during the addition of reagents can be used to adjust the detection signal obtained at the end of the assay. As an illustration, in the case of e.g. an immunosorbent assay, the current measured during sample loading, washing steps or reagent additions varies from microstructure to microstructure, and the signal obtained at the end of the assay is very likely to be different from microstructure to microstructure. Indeed, the variation of the measured current indicates that the flow rates were not equal in all microchannels, nor, possibly, in all steps of the assay. As a consequence, the time of residence of the molecules in the microstructures varies, which also generates variation of the final values obtained for the assay. With electrochemical control of the fluidics, it is then possible to correct for these variations and hence to improve the accuracy and the repeatability of the assays.

In this manner, the apparatus and methods of this invention provide a means for conducting analysis with an internal calibration of the assay. As an example, samples with slight changes in the viscosity shall flow within the microstructure at different rates; similarly, solutions may be pumped or pushed within the microstructure at various rates depending on the precision of the microfluidic control unit. One great advantage of the present apparatus is that these variations can be monitored by means of the electrochemical unit. The final result of the analysis can thus be corrected by taking account of the microfluidic variations monitored electrochemically during the various steps of the assay. Such measurements and the subsequent data processing therefore provide an internal calibration, which greatly improves the accuracy and the repeatability of the analyses.

The microchips may also contain means for temperature control, for minimisation of electronic noise and for minimisation of evaporation.

Prior to use of apparatus according to the invention, the reagents are dispensed into a microchannel or into an array of microstructures. The tips of the microchips composing the microstructure inlets are immersed in wells or reservoirs and the fluidic control system allows the filling and/or the emptying of the microstructure(s) with the reagents. Using this technique with embodiments possessing a plurality of microstructures, all the microstructures may be filled with the same or with different reagents simultaneously, and sample-to-sample contamination risks are thus limited. For some applications, the microstructure tip(s) can be integrated in a reservoir in which the sample can be loaded.

The system can be used to perform reactions or assays in the microchannels. It can be employed in the presence of a molecular phase in solution or attached on the surface of the microstructure or on a solid material integrated in the microstructure, for example a membrane, a filter, beads or the like.

Depending on the reaction or assay, detection can be performed using various principles. The transducer which is necessary for signal measurements can be placed in close contact or even integrated in the microchips.

The term “microchip” as used herein refers to any system comprising at least one miniaturised structure (or microstructure) which is a reaction or separation chamber or a conduit like a micro-well, a micro-channel, a micro-hole and the like, not limited in size and shape but enabling micro-fluidic manipulations. In the present invention, at least one such miniaturised structure(s) comprises at least one electrode so as to perform electrochemical assay(s) (as defined below). The electrode is connected to the fluidic control apparatus and used for different electrochemical events (as described below). In all cases, the electrode may serve to check whether or not the channel is filled homogeneously during the sampling and/or assay steps and to control whether each channel is empty or if change in solution has been made during a multi-step experiment. Important parameters such as the flow rate can be controlled at any time during the assay by electrochemical means. In that sense, the presence of the electrode as connected to the microfluidic control unit is unique and provides various advantages over similar approaches using optical detection and where the flow rate cannot be monitored as precisely.

The term “microchannel” as used herein refers to a single microchannel, an array of microchannels or a network of interconnected microchannels, not limited in number or shape but being sealed and having a cross section enabling microfluidic manipulation.

The microchips and microchannels are preferably disposable and may be fabricated from various materials, for example glass, quartz, polymer (e.g. polyethylene, polystyrene, polyethylene terephthalate, polymethylmethacrylate, polyimide, polycarbonate, polyurethane or polyolefines), a series of polymers or any combination of the aforesaid. They may also contain supplementary elements such as, but not limited to, membranes, chambers with beads, solid phase, sol-gel, electrodes, conducting pads or coils to control temperature and/or electrokinetic flow. The electrodes may be used to perform electrochemical measurements or to apply a high voltage for transferring the sample to a mass spectrometer by an electrospraying technique.

The term “tip” is intended to refer to the extremity of the miniaturised structure(s) contained in the micro-chip, from which a sample is either loaded into the miniaturised structure or dispensed out of the miniaturised structure. The term “connection end” (also referred to as “connection extremity”) is intended to refer to the second extremity of the miniaturised structure which is connected to the microfluidic control unit of the apparatus of this invention. For clarity, in the case where the miniaturised structure is a microchannel, the tip refers to either the inlet or the outlet of the microchannel that is not connected to the microfluidic control unit (also referred to as “pipetting device” in relation to some embodiments). The tip can be fabricated with different geometrical features such as to have a micro-channel entrance in the direction of the microchannel or perpendicular to it or at the side wall of the microchannel; it can be immersed in a reservoir or be surrounded by a fluid reservoir; finally, the tip is preferentially made of the same body as the micro-chip itself, without extension to external capillary or connection system.

The term “microfluidic control unit” or “pipetting device” means a device comprising tubes or capillaries and enabling the generation of non-turbulent molecular flux, by convection, migration or a combination thereof; the connection between the micro-chip and the microfluidic control unit can be made by clamping the microchip so as to place the microfluidic connections in aligned position with respect to the connection end(s) of the microstructure; the microfluidic control unit provides a means capable of generating a flux of molecules by controlled pulling or pushing of solution and/or to block the solution in the miniaturised structures when this is necessary during a reaction or a waiting time. The microfluidic connection unit may also be advantageously coupled to solution reservoirs containing the reagents necessary to perform a reaction or an assay, as well as blocking agents, buffers, washing solutions and the like.

The term “electrochemical assay” shall mean any electrochemical experiment using electrical conductivity and/or power in order to perform a reduction, an oxidation or an ion transfer reaction, or to perform conductimetry and/or impedance measurements, or to generate an electric field in a solution, as for instance to perform ionophoresis or patch clamp measurements, or to induce electro-osmosis or electrokinetic pumping or to generate an electrospray as may for instance be used to transfer molecules from the tip of a miniaturised structure into a mass spectrometer.

The apparatus of this invention also comprises an “electrochemical unit” which is the electronic apparatus required to perform any of the above-mentioned electrochemical assays. It may for instance include conductive pads allowing electrical connection between the solution present in the miniaturised structure(s) and the device used to perform the electrochemical assay (for example, a potentiostat, a source of controlled electrical power, an impedance measurement unit, and the like).

The core of the present invention is the combination of the above elements to perform accurate electrochemical assays in microchips: a miniaturised structure comprising a tip means to load and/or dispense a sample, as well as a connection to a microfluidic control unit, and at least one electrode connected to the electrochemical unit permitting the carrying out of electrochemical assay(s).

In some applications, an electroactive species may be advantageously added to the sample solution in order to follow the microfluidics by generation of an electrochemical signal, for example the current resulting from the reduction and/or the oxidation of this electrochemical species or the resistance along the microstructure. This may be advantageously used to provide an internal calibration of the analysis performed with the present apparatus, since the final results may be corrected according to the variations of the electrochemical signal measured during the microfluidic steps of the assays.

The apparatus of this invention may also be advantageously connected to or even integrated within a computer, thereby allowing on-line data processing and computerised control of the assays or reactions.

This apparatus is preferentially used to perform biological or chemical analysis or reactions, such as but not limited to any kind of mass spectrometry measurements, in vitro and in vivo diagnostic assays, all sorts of affinity or toxicological assays and of physico-chemical characterisations, or combinatorial synthesis of compounds.

### DETAILED DESCRIPTION OF THE INVENTION

The invention is hereinafter described in more detail by way of example only, with reference to the attached figures, in which:

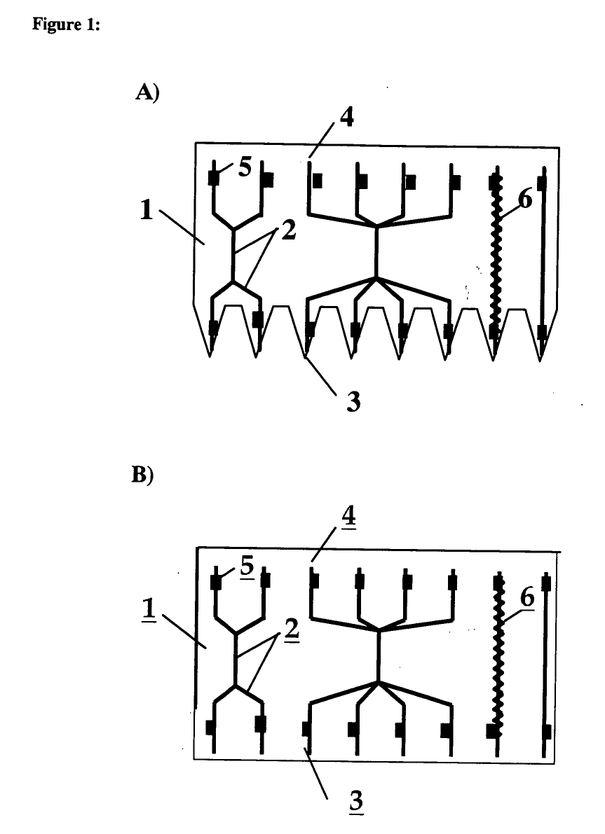

FIG. 1 is a schematic representation showing some examples of microchips and microchannel structures and connections according to the invention;

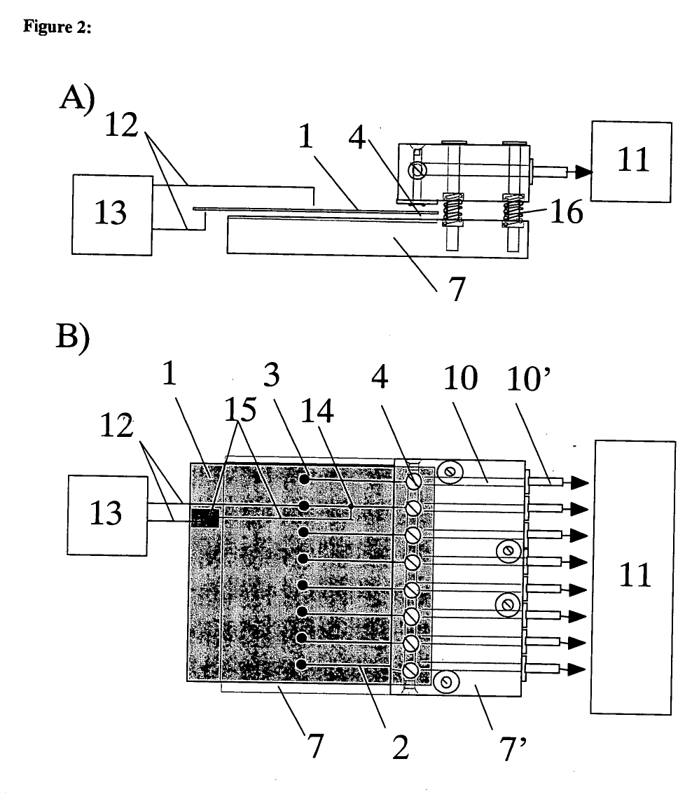

FIG. 2 is a schematic representation showing a side view (A) and a plan view (B) of an embodiment of apparatus according to the present invention;

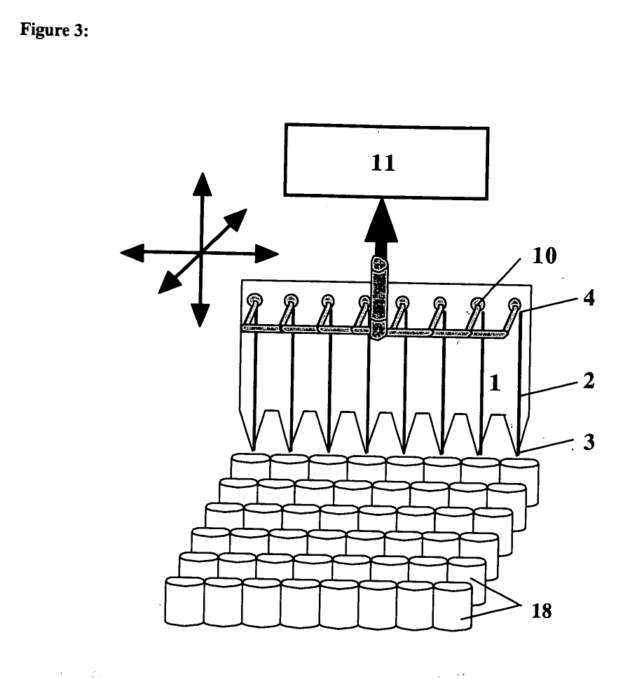

FIG. 3 is a schematic representation of an embodiment of apparatus according to the invention, comprising a series of microchannels connected with an automated system allowing both the aspiration of the reagents and the displacement of the microchips in x, y and z directions;

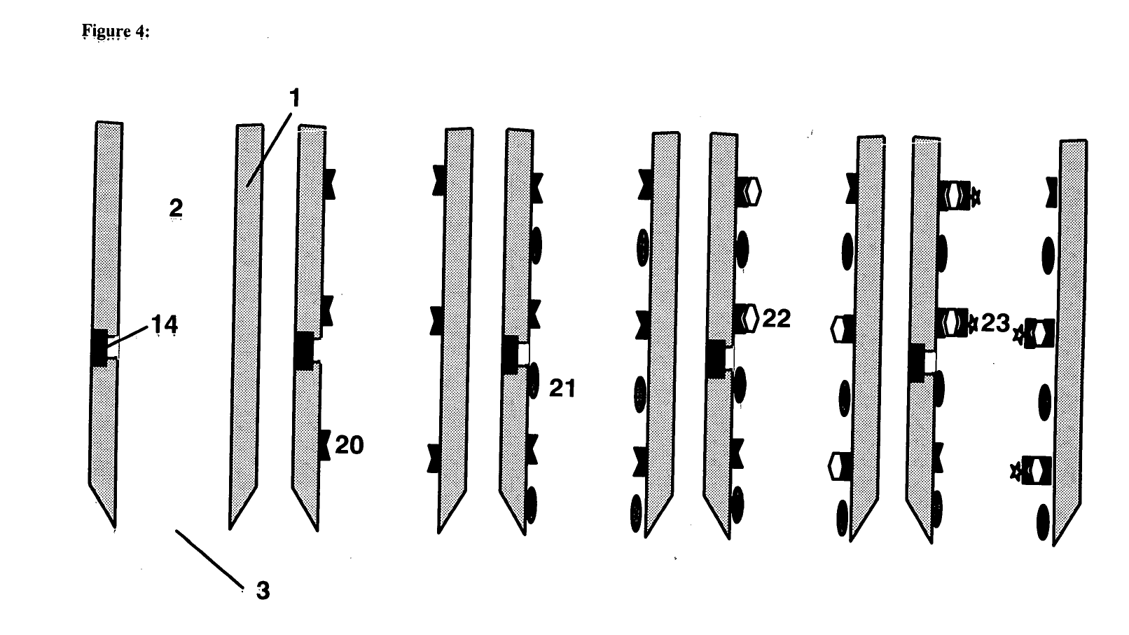

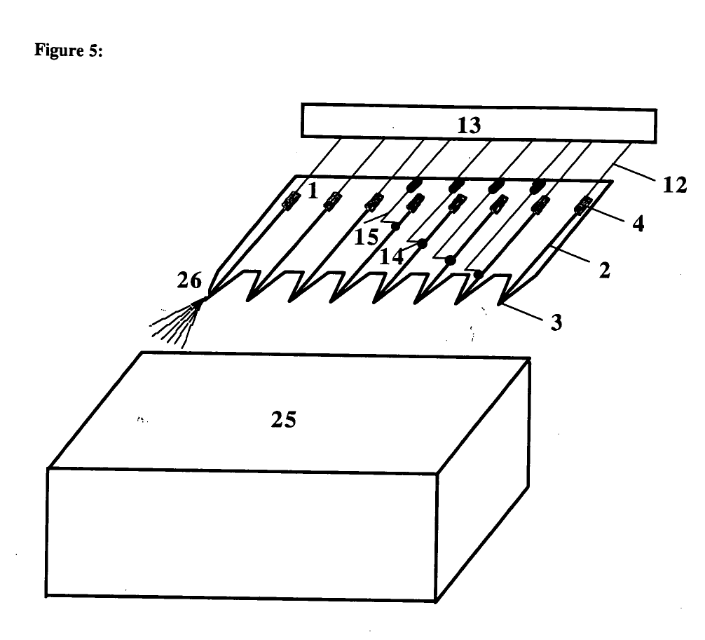

FIG. 4 is a schematic representation of the principle of a sandwich immunoassay performed in a microchip placed in an embodiment of apparatus according to the invention;

FIG. 5 is a schematic representation of the interfacing of a series of microchannels with a mass spectrometer using an embodiment of apparatus according to the invention;

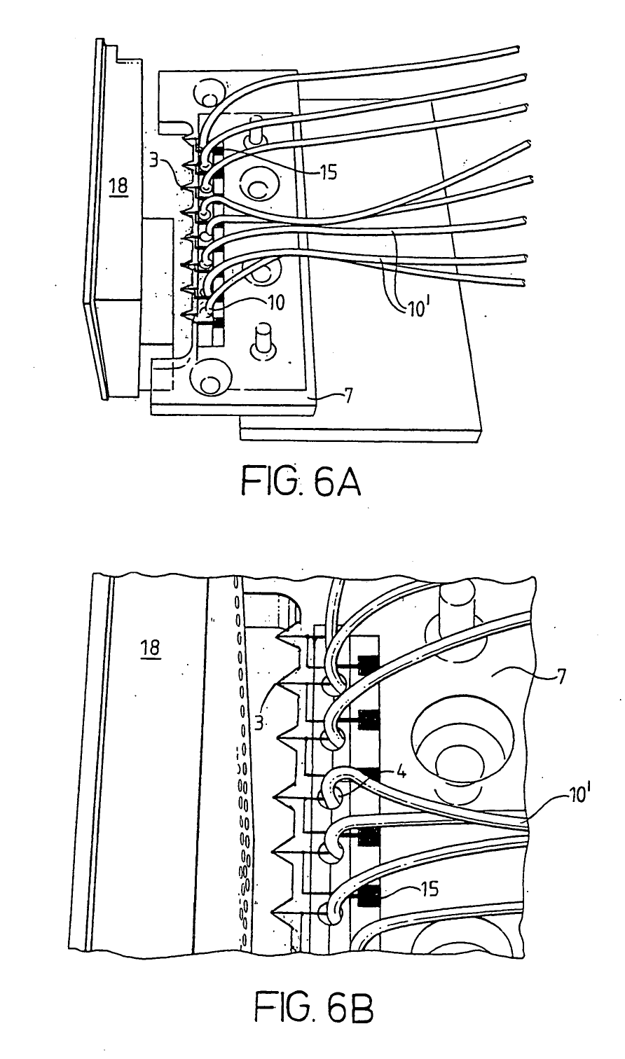

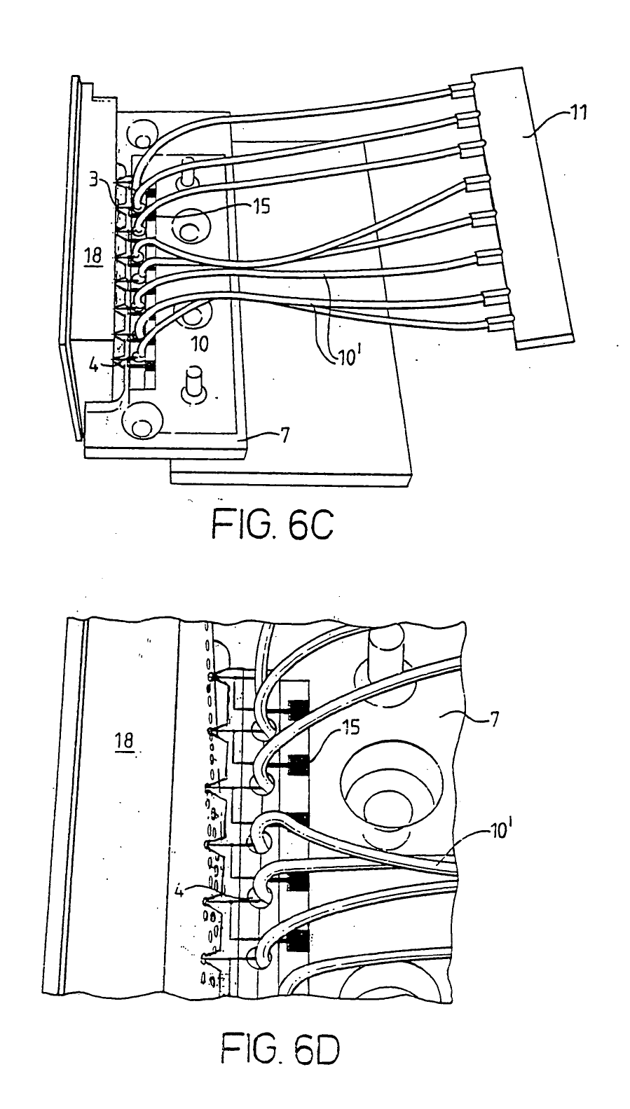

FIG. 6 is a series of photographs of an embodiment of apparatus according to the present invention which is used to take a sample placed in solution reservoirs 18 (here a microtiter plate); FIG. 6A shows a general view of the apparatus with the microchip comprising a series of 8 microstructures that is supported in a Plexiglas system enabling connection to the electrochemical unit (not shown) by way of the electrical pads 15 integrated on the microchip, as well as connection to the microfluidic control unit (only partially shown) by way of small connecting holes 10 and tubings 10′; FIG. 6B shows a closer view of the microchip and the connection systems to the electrochemical and microfluidic control units; FIGS. 6C and 6D show the same parts of apparatus as in FIGS. 6A and 6B, but in position where the microstructure tips 3 penetrate into the solution reservoirs in order to take the desired samples;

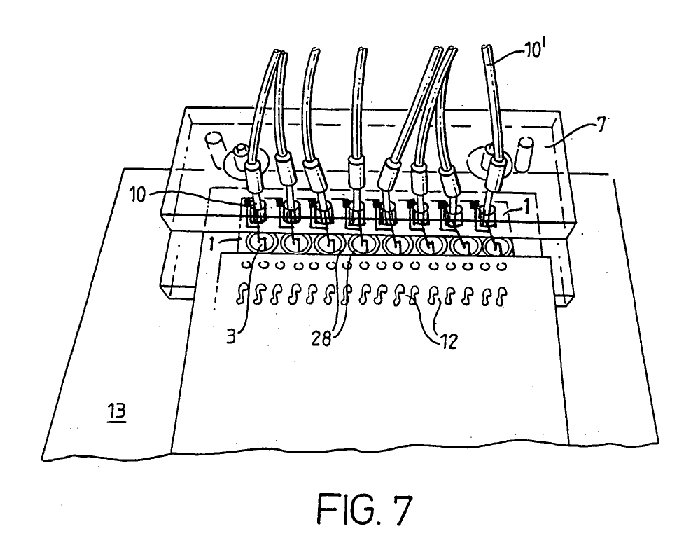

FIG. 7 is a photograph of an embodiment of apparatus according to the present invention, which comprises a microchip having microstructure tips at the top of the microchip and surrounded by reservoirs;

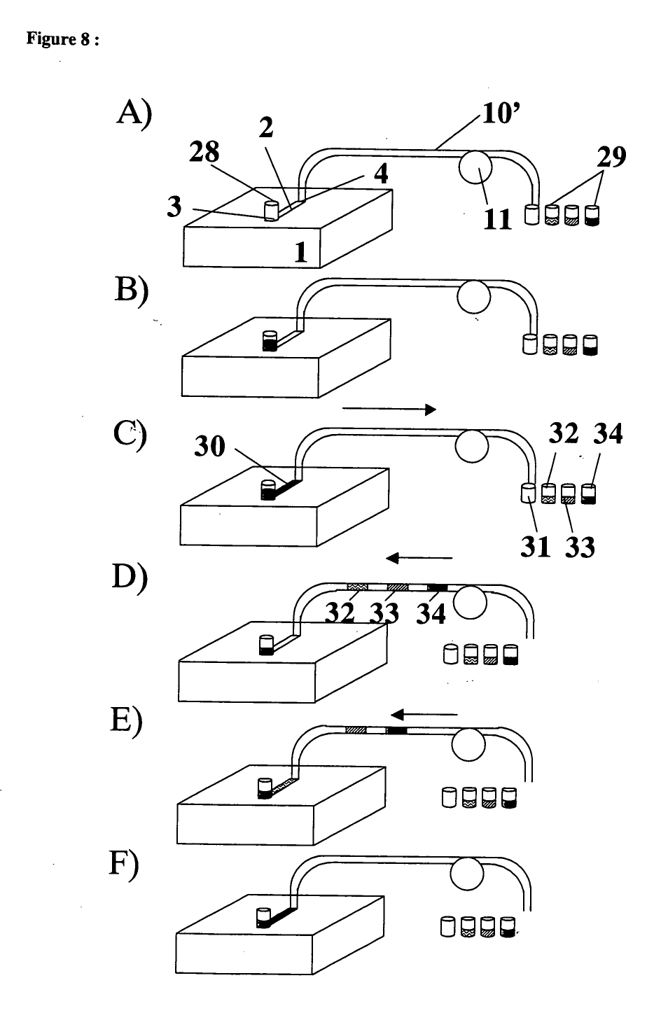

FIG. 8 shows the operation sequence of a multi-step assay performed with an embodiment of apparatus of the invention, comprising the steps of: A) connecting a microchip having a microstructure tip surrounded by a reservoir to an electrochemical unit (not shown) and to a microfluidic control unit 11 from which various solutions or even air 31-34 can be pumped, aspirated or blocked in the microstructure; B) loading a sample in the solution reservoir 28; C) filling the microstructure with the sample solution either by capillarity or by aspiration using the microfluidic control unit, and eventually letting the sample solution incubate within the microstructure; D) emptying the microstructure by pumping either air or a solution 31 into the microstructure, thereby expelling the sample solution into the reservoir 28 and filling the connection tubes 10′ with one or a series 32-34 of solutions; E) dispensing these solutions into the microstructure; F) performing an electrochemical assay (either during the pumping of one or all solutions 31-32 within the microstructure or upon blocking one, or, sequentially, each of these solutions within the microstructure);

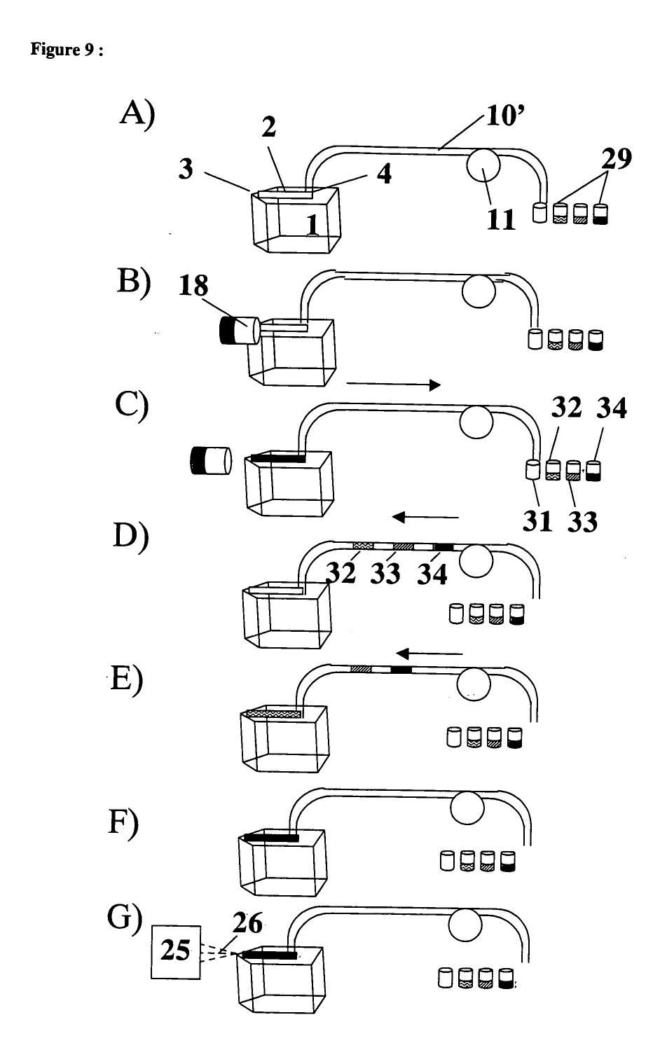

FIG. 9 shows the operation sequence of a multi-step assay performed with an embodiment of apparatus according to the invention, similar to the sequence shown in FIG. 8, but where the microstructure tip is put in contact with the sample solution and, optionally, where the final step consists in dispensing the analyte solution into a mass spectrometer 25 by generation of an electrospray 26; and

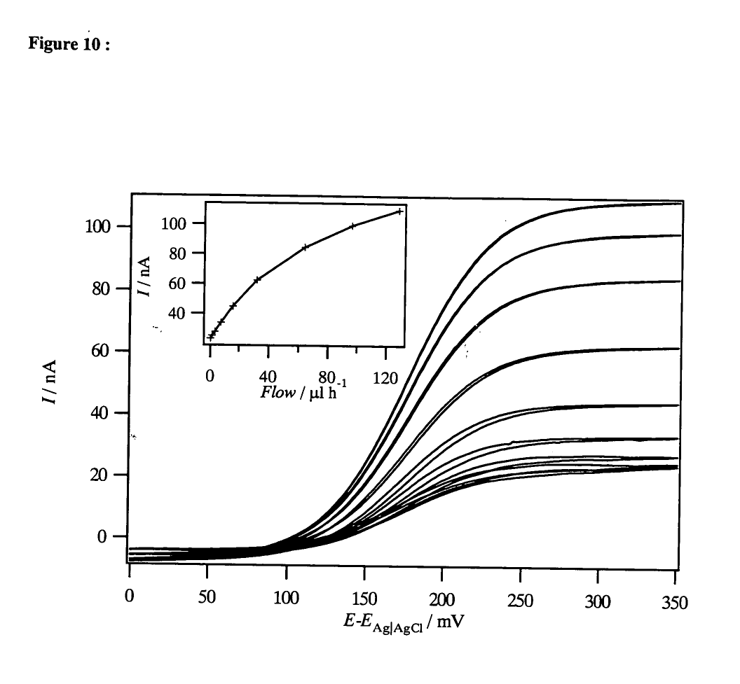

FIG. 10 is an example of the result of an electrochemical assay performed with an embodiment of apparatus according to the invention, showing how electrochemical signals can be used to determine the accuracy of the solution flow controlled by the microfluidics control unit. This figure shows the cyclic voltammetric evolution of the detection of 500 μM of ferrocene methanol under forced convection with the microchannel presented in FIG. 3a at 10 mV/s; the insert shows the evolution of the plateau current at 300 mV versus the flow rate between 0.2 and 128 μL/h.

The basic concept of the invention can be understood with reference to the attached figures, from which the various embodiments of the invention are detailed hereinafter. It is to be understood that each of the channels presented in the figures has an integrated electrode such as to enable flow control as described herein. For clarity purposes, the electrodes are not always illustrated.

FIGS. 1A and B shows an example of microchip 1 with various microchannel shapes of miniaturised structures 2 (single micro-channels and networks of interconnected microchannels). FIG. 1A shows the situation where the chip is cut in a triangular shape with the extremity in the edge of the microchannel and 1B shows the chip with an extremity of the channel on the side of the microchannel. Each of these microstructures contains one or a plurality of tips 3 and connection extremities 4. One of these microstructures shows an integrated electrode 5, whereas another of these microstructures shows integrated coils 6. Tip extremities of the microchips contain the microchannel inlets. This figure also shows how some electrodes 5 and coils 6 can be integrated in the channels.

The network of microchannels on the left hand side illustrates that two microchannels can be put in contact in order to perform separation and/or reaction of two solutions that have been pumped simultaneously from the microfluidic tips. As shown in the centre of FIG. 1, more than two microchannels are converging into a contacting zone enabling separation and/or reaction. In some embodiments, the micro-fluidic tips are not disposed on the same plane but are made in a multi-layer body that allows disposition in the three dimensions.

The microchannels may also have different surface properties to avoid or favour the adsorption of some compounds on the walls.

The microchannels may also be modified with some porous compounds, as e.g. polycarbonate membranes, microporous Teflon or other polymers, allowing the specific diffusion of gas or liquid. This can for example find applications when the reactions or the assays performed in the microchannels produce gas that needs to be eliminated, or when ion transfer experiment at the interface between two liquids have to be performed (one phase being for instance supported within such porous membrane). Also, membranes to separate physically two solutions or phases can be advantageously integrated in the microchip device. In addition, such porous material may also be used to purify a sample by adsorption of a compound present in the sample.

In the present invention, the fluidic control system may be, but is not limited to, an aspiration system (e.g. involving mechanical or pressure pumping), a capillary force flow device or an electrokinetic driven flow device. The fluidic control device may allow the filling and/or the emptying of the microchannels. The fluidic control system may be connected with an automated device allowing the sequential displacement of the microchips in x, y and/or z directions. In another embodiment, the fluidic control device may also be a simple micropipette allowing mechanical pumping and manual displacement of the microchips.

In some embodiments, the manual or automated displacement device may allow modification of the orientation of the microchannel(s) in order to change the exposition angle of the tip extremity(ies) of the microchannel(s).

FIG. 2 shows a schematic representation (A: side view; B: plan view) of apparatus according to the present invention. The microchip 1 comprises an array of eight miniaturised structures, each being composed of a micro-channel 2, a tip 3 and a connection extremity 4. This microchip is placed in a holder 7 that is manufactured to enable the precise alignment of the connection extremities 4 to the microfluidic control unit 11 by way of conduits, tubes and/or capillaries 10, 10′. The apparatus further comprises electrical connections 12 that allow connection of the electrochemical unit 13 to the electrodes 14 integrated in the miniaturised structures and the electric pads 15 disposed in the microchip (these electrical connections are shown for only one of the eight microstructures).

In one embodiment, a sample solution may be loaded into the microstructures of the apparatus by depositing a drop of solution onto each microchannel tip 3. The microchannels 2 are then filled by capillarity or by aspiration using the fluidic control unit 11 (after having clamped the connection support 16′ onto the microchips by application of a pressure onto the springs 17 in order to induce etancheity).

Then, the sample solution may be retrieved out of the microstructure using the microfluidic control unit (for instance by aspiration or pumping of air or of another solution). The microstructures may then be filled and emptied again in order to perform further analysis steps.

In another embodiment, the sample solution may be introduced into the microstructures by pumping using the microfluidic control unit, so as to be able to control the flow rate during such sample introduction. Then, the tips of the microstructures are either used as interfaces to waste reservoirs or as dispensing systems.

The electrochemical unit may also be used at any step of the filling, emptying or blocking of the sample solution in the microstructures in order to perform an electrochemical assay. In some applications, the electrochemical assay (e.g. reduction or oxidation of an electroactive compound, or conductivity or impedance measurements) is performed during all the filling and emptying steps of the analysis in order to obtain a signal measuring the proper control of the microfluidics in each microstructure.

In another embodiment, the apparatus is used to control the filling of the sample within the microstructure. To this end, the chip may be advantageously placed in the apparatus before the tip enters into contact with the sample. In this case, the microstrure is already connected to the microfluidic control unit prior to application of the sample. As the microchip is tightly connected to the microfluidic control unit, air is blocked within the microstructure and cannot escape (no venting possibility). In this manner, when the microstructure tip is put in contact with the sample, this sample cannot fill in the microstructure (no capillary fill can occur), and this can be checked thank to the integrated electrode and the electrochemical unit. In order to let the sample fill in the microstructure, it is necessary to apply a back pressure by means of the microfluidic control unit. In another embodiment, the microchip may also be disconnected from the microfluidic control unit (for instance by actuating a clamping system used to ensure fluid-tight connection between the microstructure and the microfluidic control unit), so that air becomes liable to escape out of the microstructure through its connection end, thereby enabling filling of the microstructure by capillarity. Once filled, the microfluidic control unit is connected again so as to either block the sample within the microstructure or pump or push this sample and/or other solutions. Such control of the filling of sample is very helpful to precisely fix the start point of a reaction (i.e. time equal to zero), which is crucial for the accuracy of experiments that depend on the reaction time (as for instance in enzymatic tests). This blocking method using the apparatus of this invention allows to improve the accuracy of the assays and its repeatability.

In a further embodiment, the chip may have a hydrophobic barrier to prevent the capillary fill of the sample. This will again be controlled by the electrode placed inside the microchannel. In this specific case however, the microchip does not need to be connected to the microfluidic control unit during the application of the sample to the microstructure tip.

In some embodiments, the microfluidic control unit is used during the analysis in order to block an analyte solution within the microstructures. The electrochemical unit may then be advantageously used to induce a molecular flow by application of a potential; in such analysis, the apparatus of this invention may thus be used to perform electrophoresis experiments.

FIG. 3 shows how the microchips can be connected with a microfluidic control unit 11, which is here a semi-automated aspiration system similar to a pipeting device, allowing the dispensing of the reagents into the microchannels 2 and the displacement of the microchips 1 in the x, y and z directions. The tips of the microstructures 3 are sequentially immersed in a series of solution reservoirs 18 (represented here as the wells of a microtiterplate) containing various reagents, buffers and/or washing solutions. The microchannels 2 are thus successively filled with the reagents, buffer and/or washing solutions necessary for the reactions or the assays.

In a preferred embodiment, the invention can be applied to the combinatorial chemistry field, whereby molecules are grafted onto the surface of the microstructures and combined with other molecules for the synthesis of new compounds which are then released and analysed.

In some embodiments where the reactions or the assays performed in the microchannels are endothermic, the tip may be heated by incubation of the microchips in a thermostated chamber or by passing current through the integrated electrodes or coils, as schematically shown in FIG. 1. Conversely, the temperature of the solution may also be decreased in order to stop the reaction.

In some embodiments, the invention can be used to perform homogeneous or heterogeneous (bio)chemical assays in the microchannels. These assays may involve a highly specific (bio)recognition element such as, but not limited to, an enzyme, antibody, antigen, hapten, nucleic acid, oligonucleotide or peptide. The (bio) recognition element can then be used in solution. Covalent binding may also be achieved in the microchannels with chemical compounds that allow specific (bio) recognition. In this case, the reagents necessary for the assays may be placed in an ELISA plate before measurements. The microchannels can thus for example be used to perform homogeneous or heterogeneous immunoassays.

The microchannels may also contain specific features for performing separation and/or purification. To this end, at least a portion of the microchannel may contain a covalently or physically adsorbed compound or a heterogeneous phase (like a gel, a membrane, beads and the like).

FIG. 4 summarises the principle and the successive steps necessary to perform a sandwich immunoassay in microchips 1 incorporating at least one electrode 14, as used in the present invention. The microchannel 2 is first filled with a solution of antibody 20 specific for the analyte. The antibody is thus adsorbed on the walls of the microchannels. The surface is then blocked by incubation of blocking agent 21 (e.g. a solution of BSA). This blocking agent adsorbs on the sites of the channel walls that remained free after adsorption of the antibody 20. This prevents the non-specific binding that could occur in the following steps of the assay. The samples to be analysed are then incubated, which leads to the binding of the desired analyte 22 with the antibody 20. The last step involves incubating a labelled conjugated antibody 23 specific for the analyte. Between each step, the channels are normally washed with water or buffer solutions in order to eliminate the non-fixed compounds. The detection of the sandwich complex can then be performed. Different detection principles can be used depending on the (bio)chemistry of the assay. During the steps preceding the detection of the sandwich complex, an electrochemical assay is performed in order to determine the efficiency of the microfluidic control unit. For instance conductimetry measurements allow an assessment of whether the entire microstructures are filled with solution; similarly, amperometric measurements may be performed in order to assess the efficiency of the various steps of the assay.

The assays or the reactions performed in the microchannels can be detected using various principles such as, but not limited to, luminescence (fluorescence, UV/Vis, bioluminescence, chemiluminescence, electrochemiluminescence), electrochemistry or mass spectrometry.

In some embodiments, the microchips are interfaced with a detector placed outside of the microchannels. In this case, the detector can be for example a photomultiplier tube or a mass spectrometer.

Before the detection step, the solution contained in the microchannel can be subjected to a purification and/or separation step (for example using chromatography, selective membranes, filters or electrophoretic separation).

FIG. 5 shows how the tip ends 3 of microchips 1 can be interfaced with a mass spectrometer 25 for the detection of a molecule. After completion of, for example, an immunological reaction in the microchannels 2, the complex is desorbed and eluted. The tip extremity 3 is then used to inject the eluate into the mass spectrometer by generation of an electrospray 26. To this end, the solution must be in contact with an electrode and to an electrochemical unit that serves to apply a high voltage between the microstructure and the mass spectrometer. FIG. 5 shows such an electrode 14 which may be placed at various positions in the microchannels or in the connection extremity 4 of the microstructure. When this electrode is integrated in the microchannel, a conducting pad 15 is preferably directly manufactured on the microchip; the electrode is then further plugged into the electrochemical unit by way of electrically conductive connections 12 (e.g. screened cables).

In some embodiments, the detector can be integrated in the microchannels. In this case, the transducer may be for example an electrode or a photodiode.

In other embodiments, the microchannel tip is not used to fill in the microchannel with the solution of interest but is used to dispense the solution out of the microchannel into another separation, purification or detection apparatus. To this end, the microfluidic control unit allows control of the volume of solution dispensed from the microstructure tips. For instance, the microchannel can be used as an electrospray interface for MS analysis. In another embodiment the microchip can be placed horizontally and a series of solution reservoirs (e.g. a microtiter plate) can be placed vertically such as to enable easier sampling into the microstructures and then dispensing of the solution into the mass spectrometer.

FIG. 6 presents several views of an example of apparatus according to the present invention, in which solution reservoirs 18 are placed in contact with the microstructure tips 3 in order to fill a series of microchannels with analyte solutions. It is straightforward that either the microchip or the solution reservoirs may be displaced in all x, y and z directions. The microchip supporting the microstructures is placed in a holder enabling interfacing with the electrochemical and the microfluidic control units (not shown) by way of electrical connections 15 and tubings 10′. In this case, the microchip can incorporate a solid phase such as to enable desalting, specific affinity assay or other sample preparation. A solution of spray composed for example of methanol, acetonitrile and acidic solution may be stored in the tubes 10′ and can serve to desorb samples that have been previously immobilised in the microchip. In one embodiment, microbeads can be placed in a reservoir between the chip and the microfluidic control unit such as to enable sample pretreatment (as e.g. desalting or affinity reactions) prior to mass spectrometry analyses.

In some embodiment, the microstructure tip is an inlet on the side of the microchip in contact with the sample solution to be analysed. FIG. 7 shows an example of such microstructure tip inserted in an apparatus of the present invention. In this example, reservoirs 28 can be integrated on the top of the microstructure tips such as to enable the sample solution to be dispensed via the tips into the microstructures. The solution can then enter the microstructures either by capillary action or by aspiration from the connection extremity. In some embodiments, the microchip can be connected to the fluidic control device in such a way that capillary fill will be prevented by the back pressure insured by the fluidic control device. Only when the fluidic control device is aspirating, can the sample enter the channel. FIG. 7 also shows the electrochemical unit 13 with its electrical connections 12, which is used to perform the electrochemical assay(s) in each microstructure.

FIGS. 8 and 9 illustrate the sequence of an assay performed with an apparatus of the invention, depending on the way the sample and reagents are dispensed into the microstructures and with two different designs of microstructure tips. In FIG. 8, a reservoir is integrated on the tip end of the microstructure and ensures contact of the solution with the chip. It is notable that this reservoir can be used to receive successive solutions for performing multi-step assays such as syntheses, analyses, and so forth. In one embodiment, different reagents 32, 33 and 34 can be loaded in the non-turbulent flow connection tubes 10′ and separated with an inert solvent or even a gas bubble 31. Pumping the different reagents inside the microchip can make a reaction occur, such as but not limited to, ELISA, affinity assays, washing steps, desalting step, etc.

In some embodiments, the reagent 31 to 35 may contain beads that are pumped by means of the microfluidic control unit such as to pack them at the end of the connection tubings (10′) or at a desired position within the microstructure. These beads may have various physico-chemical properties and may also be functionalised with molecules, depending on the use of these beads. Such beads addition may for instance be advantageously used to desalt a solution, to perform an affinity reaction or to synthetise compounds by combinatorial chemistry, notably with molecules previously grafted on these beads. In certain applications, a membrane can also be placed between the connection tubings (10′) and the connection end of the microstructure (4) such as to enable filtration, or different reactions such as adsorption, desorption, desalting, immunocapture, enzymatic assay and so forth.

Integration of either beads or membrane within the apparatus of this invention is of particular interest in mass spectrometry analysis, where systematic desalting of the sample is generally required prior to injection into the mass spectrometer. The above features may thus be advantageously used in applications where the present apparatus serves for instance to inject samples into a mass spectrometer by electrospray ionisation (ESI) from the microchip or to dispense samples onto a plate devoted to mass spectrometry measurements using matrix assisted laser desorption ionisation (MALDI).

In another embodiment the assay is performed with the tip being placed in contact with the well for the sample loading.

In another embodiment, the contact between the connection extremity 4 of the microstructure and the microfluidic control unit 11 is not tight (see FIG. 2) and enables the microchip to be filled by capillary action. It is important to note here that the flow of solution should stop at the end of the microstructure. To this end, a hydrophobic layer may optionally be placed around the microstructure outlet, thereby preventing cross-contamination of the apparatus. After the filling of the sample, pressure can be applied on the upper part of the support 7′ serving as connection between the microchip and the microfluidic control unit such as to induce tight sealing and to prevent solution leakage. At this stage, a solution can be pumped towards and through the microchip without contaminating the microfluidic control unit. A succession of different analytes can then be pumped within the microstructures such as to place different solution as exemplified in FIGS. 3 and 4, as well as in the sequences of FIGS. 8 and 9. The fluidic tubing should have an internal diameter such that it may prevent generation of turbulent flows and that segments of different solutions can be pumped to the chip, said segments of solution being separated by an air bubble. For example each washing solution, secondary antibody or further reagent solutions (such as e.g. an enzyme substrate) can be preloaded in the tubes with an air bubble segment for separating them. Then, the pumping of these solutions through the microstructures allows the entire sandwich immunoassay to be performed without any manipulations and without external reagent addition.

As a demonstration of the apparatus of this invention, experiments have been carried out by connecting the micro-chip to a syringe pump serving as microfluidic control unit in order to apply a forced convection into a series of microstructures. Only one microchannel is integrated in the apparatus of the invention which is similar to that shown in FIG. 6, but with only one microfluidic connection. The microchips used here are 75 micron polyimide foils in which microstructures comprising a 100×60×10,000 μm microchannel with one tip and one connection extremity at each end of the microchannel are fabricated by plasma etching. These microstructures further incorporate gold microelectrodes and conductive tracks that are connected to a potentiostat which is the electrochemical unit used to perform the electrochemical assay which consists here in the oxido-reduction of an aqueous solution of 500 μM ferrocene methanol. The cyclic voltammetric response at a scan rate of 10 mV/s as a function of the flow rate (set between 0.2 and 128 μL/h) induced by a 100 μL syringe has been recorded and is presented in FIG. 10. The insert in FIG. 10 further shows the evolution of the plateau current at an applied potential of 300 mV versus silver/silver chloride, as a function of the flow rate. The intensity of the current is strongly dependent on the flow rate because the forced convection is constantly renewing the diffusion layer above the electrode.

1. Apparatus for performing an electrochemical assay or a reaction, using electrical conductivity and/or power in order either to perform a reduction or an oxidation or an ion transfer reaction, or to perform conductimetry and/or impedance measurements, or to generate an electric field in a solution, or to perform any combination of the aforesaid, the apparatus comprising:
at least one micro-chip, the or each said micro-chip possessing at least one microstructure having: a tip end adapted for uptake of a fluid sample into and/or discharge of a fluid sample from said microstructure; a microfluidic connection end; and an electrode integrated in said microstructure;
a microfluidic control unit communicating with said microfluidic connection end of said microstructure and adapted to push, pull or block fluids in said microstructure;
an electrochemical unit adapted to apply an electric field or a current to fluid in said microstructure and/or to measure an electrochemical event therein;
and, optionally, support means adapted to support said micro-chip(s) in relation to said microfluidic control unit in such a manner as to ensure fluid-tight connection therebetween.

2. Apparatus according to claim 1, wherein said electrochemical unit comprises a potentiostat, a power supply, an impedance or conductivity measurement device or a computer.

3. Apparatus according to claim 1 or 2, wherein said electrochemcial assay or reaction is used to monitor the microfluidics within said microstructure.

4. Apparatus according to claim 3, wherein the electrochemical monitoring of the microfluidics serves as internal calibration of the final detection signal.

5. Apparatus according to any preceding claim, comprising a plurality of microstructures provided in one or a plurality of micro-chips, permitting simultaneous electrochemical measurement in more than one microstructure.

6. Apparatus according to any preceding claim, wherein said electrochemical unit is adapted to detect a molecule by reduction and/or oxidation.

7. Apparatus according to any preceding claim, wherein said electrochemical unit is adapted to induce electrokinetic pumping of molecules.

8. Apparatus according to claim 1, wherein said microfluidic control unit comprises a pump or a pipetting system.

9. Apparatus according to any preceding claim, further comprising a valve disposed between said microfluidic control unit and said microfluidic connection end of said at least one microstructure.

10. Apparatus according to any preceding claim, wherein the or each said microchip is made of polymer, glass, quartz or a combination thereof.

11. Apparatus according to any preceding claims, wherein the or each said microchip is disposable.

12. Apparatus according to any preceding claim, wherein the or each said microchip is produced by laser photoablation, injection moulding, embossing, plasma etching, elastomer casting, silicone technology or a combination thereof.

13. Apparatus according to any preceding claim, further comprising a detector disposed outside the or each said microstructure, said detector(s) being interfaced with said micro-chip(s).

14. Apparatus according to claim 13, wherein said detector is a photomultiplier, a mass spectrometer or a nuclear magnetic resonance (NMR) system.

15. Apparatus according to any preceding claim, wherein said microstructure comprises a microchannel, or a network or array of interconnected microchannels.

16. Apparatus according to any preceding claim, wherein said microstructure is sealed.

17. Apparatus according to claim 16, wherein a polymer layer is laminated or glued to seal said microstructure.

18. Apparatus according to claim 15 or according to either of claims 16 and 17 as appendant to claim 15, comprising an arrangement of interconnected microchannels in which a plurality of microchannels converge into a single microchannel, whereby said arrangement comprises a single microstructure tip and a plurality of microfluidic connection ends, or a plurality of microstructure tips and a single microfluidic connection end.

19. Apparatus according to claim 15 or any claim appendant thereto, wherein said interconnected microchannels are not disposed in the same plane, but are fabricated in three dimensions.

20. Apparatus according to any preceding claim, wherein at least a portion of walls of said microstructure is modified by chemical, biological or physical means, by the provision of porous material or by any combination of the aforesaid.

21. Apparatus according to any preceding claim, wherein said microstructure comprises a solid phase.

22. Apparatus according to claim 21, wherein said solid phase comprises molecules, a membrane, a gel, a sol-gel or beads.

23. Apparatus according to claims 20, 21 and 22, further comprising molecules grafted onto at least said portion of walls of said microstructure and/or onto said membrane, gel, sol-gel or beads.

24. Apparatus according to claim 23, wherein said molecules are proteins, peptides, antigenes, antibodies, enzymes, oligonucleotides, nucleic acid sequences, haptens or a combination thereof.

25. Apparatus according to claim 23 or claim 24, wherein said molecules are grafted by physical or chemical adsorption, by covalent binding, or by a combination thereof.

26. Apparatus according to any of claims 22 to 25, wherein said membrane physically separates two solutions or phases.

27. Apparatus according to any preceding claim, wherein said tip is formed at the edge of said micro-chip.

28. Apparatus according to claim 27, wherein said tip has a pyramidal, a parallelepipedic or a conical shape.

29. Apparatus according to any preceding claim, wherein said tip is adapted to generate an electrospray.

30. Apparatus according to any preceding claim, wherein said tip is integrated in or is surrounded by a fluid reservoir.

31. Apparatus according to any preceding claim, wherein said tip comprises an electrode.

32. Apparatus according to any preceding claim, wherein said support means comprises a clamping system to ensure fluid-tight connection between said microfluidic connection end(s) and said microfluidic control unit.

33. Apparatus according to any preceding claim, wherein said apparatus and/or said microchip can be displaced in x, y and/or z direction either manually or by means of an automated device.

34. Apparatus according to claim 33, wherein said manual or automated device permits modification of the orientation of the microchannel in order to change the orientation angle of said microstructure tip.

35. Apparatus according to any preceding claim, further comprising a temperature control unit, an electrical isolation chamber (for example a Faraday cage) and/or a humidity-controlled chamber preventing evaporation.

36. Apparatus according to any preceding claim, wherein said electrochemical unit, said microfluidic control unit and said support means are integrated in a single platform, so as to provide a portable system.

37. Apparatus according to any preceding claim that is further connected to and/or integrated into a computer.

38. A method of performing an electrochemical assay or a reaction, using the apparatus of any preceding claim, the method comprising the steps of:
(a) placing said microchip in said support means;
(b) placing a sample in contact with said microstructure tip;
(c) filling said microstructure with said sample, either by capillary action or by pumping or aspirating said sample by means of said microfluidic control unit;
(d) using said microfluidic control unit either to pull, push or block said sample in said microstructure;
(e) actuating said electrochemical unit to perform an electrochemical assay using electrical conductivity and/or power to perform a reduction or an oxidation or an ion transfer reaction, or to perform conductimetry and/or impedance measurements, or to generate an electric field in a solution, or to perform any combination of the aforesaid.
(f) optionally, repeating above steps (b) to (e).

39. A method according to claim 38, wherein a plurality of samples and/or other solutions are introduced into said microstructure using said microfluidic control unit.

40. A method according to claim 39, wherein said other solutions are washing solutions, buffer solutions and/or reagent solutions.

41. A method according to any of claims 38 to 40, further comprising the step of adding an electroactive species to said sample(s) or said other solution(s) and monitoring the microfluidics thereof by performing an electrochemical assay(s), for example by measuring of the resistance or impedance along at least a portion of said microstructure or the generation of a current resulting from the reduction and/or the oxidation of said electroactive species.

42. A method according any of claims 38 to 41, wherein said electrochemcial assay or reaction is used to monitor the microfluidics within said microstructure.

43. A method according to claim 42, wherein the electrochemical monitoring of the microfluidics serves as internal calibration of the final detection signal.

44. A method according to claim 43, wherein a software processes the data obtained during the electrochemical monitoring of the microfluidics to perform said internal calibration of the final detection signal.

45. A method according to any of claims 38 to 44, further comprising the step of bringing said microstructure tip into contact with a solution reservoir to enable the uptake or discharge of a sample and/or another solution.

46. A method according to any of claims 38 to 45, wherein said microstructure tip is used as the disposable part of pipetting device.

47. A method according to any of claims 38 to 46, wherein the filling of the sample in said microstructure by capillary action is prevented either by means of said microfluidic control unit or by the presence of a hydrophobic barrier at said microstructure tip.

48. A method according to any of claims 38 to 47, wherein said tip is used to generate an electrospray.

49. A method according to any of claims 38 to 48, comprising the further step of injecting said sample(s) or said other solution(s) contained in said microstructure into a purification, separation and/or detection device, as for example a chromatograph, a spectrometer, a photometer, a gel, a column, a selective membrane, a filter, or an electrophoretic separation apparatus.

50. A method according to any of claims 38 to 49, wherein the assay or reaction performed in said microstructure is detected or followed using light absorption, luminescence (for example fluorescence, bioluminescence, chemiluminescence, electrochemiluminescence), electrochemistry or mass spectrometry.

51. A method according to any of claims 38 to 50, for performing chemical and/or biological analysis and/or synthesis.

52. A method according to claim 51, for use in mass spectrometry analysis.

53. A method according to claims 52, wherein said apparatus comprises means to desalt samples prior to injection into a mass spectrometer by generation of an electrospray or prior to dispense of said samples onto a matrix assisted ion desorption ionisation (MALDI) plate.

54. A method according to claims 51 or 52, for performing clinical, human or veterinary in vitro and/or in vivo diagnostics.

55. A method according to claim 54, for performing immunological assays.

56. A method according to claims 51 or 52, for performing physico-chemical assays, toxicological assays, affinity assays, microbiological assays and/or cellular assays.

57. A method according to claims 51 or 52, for performing lipophilicity measurements, ion transfer reactions, solubility assays and/or permeability tests.

58. A method according to any of claims 38 to 57, for performing synthesis by combinatorial chemistry.

59. A method according to any of claims 38 to 58, for performing fully automated analysis and/or synthesis.

60. Use of apparatus according to any of claims 1 to 37 to perform chemical and/or biological analysis and/or synthesis.

61. Use according to claim 60 in mass spectrometry analysis.

62. Use according to claim 60 or 61 to perform clinical, human or veterinary in vitro and/or in vivo diagnostics.

63. Use according to claim 62 to perform immunological assays.

64. Use according to claims 60 to perform physico-chemical assays, toxicological assays, affinity assays microbiological assays and/or cellular assays.

65. Use according to claim 60 to perform lipophilicity measurements, ion transfer reactions, solubility assays and/or permeability tests.

66. Use according to claim 60 to perform synthesis by combinatorial chemistry.

67. Use according to any of claims 60 to 66 to perform chemical and/or biological analysis and/or synthesis with electrochemical internal calibration of the final detection signal.

68. Use according to any of claims 60 to 67 to perform fully automated analysis and/or synthesis.
